(19) United States
(12) Patent Application Publication
(10) Pub. No.: US 2006/0127879 A1
(43) Pub. Date: Jun. 15, 2006

(54) Electrodynamic profiling of genomic response in the cell

(76) Inventor: Anthony Stephen Fuccione, Lynn, MA (US)

Correspondence Address: ANTHONY S. FUCCIONE, 155 OCEAN STREET, LYNN, MA, 01902, US

(21) Appl. No.: 10/749,827

(22) Filed: Dec. 31, 2003

(51) Int. Cl. C12Q 1/00; H01L 21/00; G06F 19/00

(52) U.S. Cl. 435/4; 438/1; 702/19

(57) ABSTRACT

A method of cellular evaluation based on the electronic nature of cells is reveled though cellular reproductions use of a magnetic force. The dynamic process of nuclear response is shown to be electronic in nature relative to DNA mediating electrons hydrogen bonding in bases pairing of DNA though out the a cell cycle and finally during metaphase one see the magnetic component of interaction. The electrostatic understanding of magnetic force is not well defined in physics in the process of electrodynamic. Cells use electrodynamic interaction within the cell are being studied as the basis and using the cell to measure and define electrodynamic interaction with the system that is biological a call. Specifically DNA thought the electronic interaction interactions. It appears infrared spectrum holds promises to help in revel these mechanisms. The promise of understanding or merely evaluation of electrodynamic interaction holds great promise to science with the greatest medical implication to understand genomic responses in cells. Understanding how the DNA interacts within a cell dynamic transition are known to take place and these are regulated thought electrodynamic interaction.

### CROSS-REFERENCE TO RELATED APPLICATIONS

A provisional patent was filed on Dec. 31, 2002.

Application No. 60/437,621

See attached (doc 1)

A biological cell is the model. Chemistry is used to follow the flow of electronic energy, ionic flux and physics is explain cellular reproduction.

The method uses the novel understanding of bioelectromagnetic for cellular evaluation

Bioelectromagnetic response contains broad practical application. The invention uses a novel method of cellular evaluation for research and development, commercial and industrial application the study of energy within the cell. Evaluating a cell as electronically functioning the electrodynamics of electromagnetic fields interaction with in as cell. The cell nucleus or DNA defining the electromagnetic field and electrodynamics of cell genomic function to it own reproduction. Use of physical applications include: electronics, bioinformatics, chip design, computer design on nanoscale level “nanotechnology” and usage and “biotechnological” also for use in stem cell (controlling cell differentiation), diseases, cancer research (controlling cell differentiation) and even as a clinical diagnostic enabler with immediate application focus and novel uses in electroporation, radiotherapy, drug discovery.

wouldaddanTo explore the conductance,electostatic,electronic of DNA. (see review DOC 2)
Doc 2 express DNA MEDIATES ELECTRONIC CHARGE TRANSFER these are claims( as defined by patent law) on electronic nature and abilities of DNA and the circuit of the cell. The circuit will be mimic to use for energy production and storage.
Charge mediatation in a cell shows the functional packing of DNA by histones. (DOC 4) Core histone function electrodynamic in an ordered energy and allow charge to be mediated in a super efficent manner and molecular size becomes miniaturized to the final point of non conductive as during cellular reproduction. Flexoelectric properties of DNA intercellularly and extra cellular(DOC 6)

Intercellular

With DNA, RNA, proteins, synthetic hybrids

with RNA, protiens, histone codes.

In the cellular processes of life DNA has the ability to be: superconducting, conductor, capacitor, transistor, resistor, insulator by means of confirmational (liquid crystal) transformations due to ionic flux, hydration and p.H. and known thermodynamic properties and unknown electrodyanmical properties. This allows the abilities of DNA to oscillate at frequencies(ex.bead on string) vibrational forces which can signal for modification and transformation within the cell. Moreover these vibrational forces synchronize chemical and physical pathways signaling within groups of cell retaliating information of the environmental conditions. Harmonizing (large groups of cells) function and needs within the targeted area (magntoelastic properties)

### STATEMENT REGARDING FEDERALLY SPONSORED RESEARCH OR DEVELOPMENT

Not applicable.

### REFERENCE TO SEQUENCE LISTING

This reference is enclosed below.

### INCLUDE

Gene bases or base pairing

Ordered energy of bases and ionic interaction . . . p.H

Amino acid interactions RNAs.

### BACKGROUND OF THE INVENTION

The research question is how does long term exposure to low—perhaps minute—levels of electrical current affect the body? The research question is how does long term exposure to low—perhaps minute—levels of electrical current affect the body?

The background for the invention presents a classical theory and methodology of biological and physics evaluation of energy usage related to electromagnetic electronic and magnetic interaction with in a biological system—the cell.

Although traditionally biologist have viewed the simplest biological system, the cell, based thermodynamic understanding, however to understand genomic function Electrodynamics deal with the electronic nature of the cell. (8)

Within a cell the structural transition of DNA mediates functional changes in the permitivity of the cells' plasma membranes ionic channels resulting in cytosolic pH. change. DNA intracellularly orchestrates electrochemical gradients functioning to direct bioelectromagentic fields mechanistically controlling the cell.

“The Incredible Life and Times of Biological Cells” by Paul Nurse, states that “ordered signaling patterns can emerge from relatively simple wiring diagrams and rules of operation”(1) The cell during reproduction as birefringement of chromatin during prophase displays an electronic and mechanical relationship though out metaphase, teleophase, anaphase. The physics of this process can be explained by electrostatics(2), yet the process and understanding is static. The process of mitotic division is dynamic and an electrodynamic view (completes and explains the movement of a magnetic field.(3) The middle of cellular reproduction metaphase is a magnetic field.

The process of mitosis/meiosis can be explained and shown as the formation and deformation of a magnetic field and considering a magnetic field as “a rule for operation”. Viewing the cell as an electronic structure, simply as a battery, and the controls being bioelectromagnetic. The capacitances of the cell (battery) electronic properties are based nuclear structural confirmations of the cellular DNA.

The intrinsic structures of cellular DNA orchestrates biochemical synthesis using electrochemical, (electronic: ionic transduction across the plasma membrane and based in a tensgrity model(7), chemical: inducing cystolic pH. changes), the symmetry of the (electron) energy and current densities are controlled and displayed by the electrodynamics and conformational transitions of nuclear architecture DNA.(double helix, bead on string, lampbrush, chromatin).

Ionic currents or “asymmetries of ionic flux” (4) though the plasma membrane H2O,(Na, K, Ca2+,Mg2+) act in response to ordered energy and upon intracellular concentrations vary (flux) the nuclear (DNA) architecture during the cell cycle or “embryo patterning.” (4) Base pairing, AT, GC banding and functions of the electronic cell are sequence dependent.

Variable p.H. intracellular conditions (ATP/cyclins, ADP/cdk, AMP/cdk2) are controlled by the electrodynamics of the DNA structure with regards to the conductance and function of the cell.

The Bioelectromagnetic field is produced before replication intracellularly by the downward spiral of phospholations and the upward composition of DNA. The highest ordered state of DNA is chromatin at metaphase that displays the bioelectromagnetic mechanism.

2) www.crab.rutgers.edu//˜gagliard/webpre.pdf

3)http://micro.magnet.fsu.edu/electromag/java/mitosis/index.html

4)http://www.drmichaellevin.org

5) www.cancerci.com/content/2/1/3

6)http://www.physics.brown.edu/Users/Faculty/valles/VallesLabFiles/BFieldManipulation.htm

7) http://www.hms.harvard.edu/dms/bbs/fac/ingber.html

8) www.powerlinefacts.com/goodman-blank.pdf

background (what>problem you are addressing, what progress has already been made in this>area by mainstream science),

What is the electronic nature of a cell?

A theory which brings the biological system a cell, though chemical explanation (acid/ base reactions, Hydrogen ion flux), electron (ic) movement revealing a physics (al) relationship

Although more today mainstream science crossing over the disciples of the hard sciences is more acceptable, specialization of intricate study prevails and whole systems (systems biology) are disregarded. Moreover, the physicist does not fully understand biological system, as the chemist and the biologist trained in all disciples' follows only the laws of them. The biological cell ultimately has been viewed in thermodynamics for the last century; however, electrodynamic interactions drive the electronic nature of DNA and the cell.

Many questions remain in cell biology today. These can be addressed as we offer answers to unexplained biological metabolic pathways as genomic function controlling cellular response. The dynamical process of life in simplest form is a cell. Most basic to life is cell reproduction: as one cell divides into two cells. The process of cellular reproduction the controls and mechanism by which this vital process takes place remains poorly understood. To address this fundamental question which remains unanswered viewed in broad basic trinity scientific understand yields unparalleled explanation of cell function to the mechanistic controls. Basic scientific understanding in a trinity of the hard sciences: biology, chemistry and physics leads to explanation.

Bioelectromagnetics Society (BEMS) being the most closely associated with the mainstream science in technological understanding. The group largely focuses on emf interactions Do cell phone cause cancer? Do powerlines cause cancer Magnetic therapy and alike. BEMS fails to understand bioelectromagnetic interactions ag described here. The model present model helps explain electromagnetic field interaction during cell cycle (5) by Whealtey et al call for a plausible hypothesis. J. Barton of University of California as she present controls of electron transfer in DNA, understanding electron transfer is critical is the cell system. Fritz Popp presents a “cavity resonator” which is a good model, yet measures photon emission. Biophotons are measuring photons herein we examine electronic usage.

Using biology as the system: a single cell metabolic pathways, chemistry as the central science acid/base reactions to show ionic (electric) flux and physics study of electromotive force in relation to magnetic fields and electrical interactions, gives a picture that helps answer many questions. The trinities of these sciences and there relationship have been understudied.

“The search for fundamental relationships between charge transport phenomena and magnetic exchange coupling provides a strong driving force for our research. We apply the tools of physical-organic chemistry to gain insight into the structural and electronic factors.”

“there are not many individuals looking at these relationships.” Dr. Natia Frank of University of Washington

A model is presented within a biological system the cell and the relationship of charge transport (ionic flux) explained though chemistry, which displays the physics of magnetic field . . . Bioelectromagnetic.

The center of the cell is the nucleus, basis of chemistry is ionic flux, and physics shows electronic energy as a magnetic field.

During metaphase all cells stop as chromosome align in the middle the cell. To impinge the resemblance of the cell to a magnetic field. Is mitosis at metaphase a magnetic field? (The model is called 3M mitosis metaphase magnetic). Could a magnetic field direct cell activity?

Within the cell does DNA function as a “magnetostrictive and magnetolelastic” device? Giving DNA “sensory capabilities including liquid, temperature, viscosity, density, sound, force, movement pressure, vibration, light chemical analyte consetrations. The sensory can be measured physically or remotely.(energy doc 7 intro pg2.) (Flock)

a simple explanation of your theory,

The process of mitosis/meiosis can be explained and shown as the formation and deformation of a magnetic field and considering a magnetic field as “a rule for operation”. Viewing the cell as an electronic structure, simply as a battery, and the controls being bioelectromagnetic. The capacitance of the cell (battery) electronic properties are based nuclear structural confirmations of the cellular DNA.

Explaining simple and logical scientific solution giving ability to view the biological cell in a novel way as having and electronic structure and nature. The theory explains the mechanistic function of the genome, though a basic unexplored understanding, which brings together biology, chemistry and physics. “It sounds very interesting” Robert Langer MIT.

“The Incredible Life and Times of Biological Cells” by Paul Nurse, states that “ordered signaling patterns can emerge from relatively simple wiring diagrams and rules of operation”(1)

The simple diagram pointed to is metaphase and the magnetic field. These show the rules of operation. This links bioelectronic magnetic phenomena to cell division(10)

I suggest that cellular reproduction controls are magnetic. During metaphase we can see the final formation of the electrically induced magnetic field and the deformation of the field pulling chromosomes to respective inversely proportionality to respective poles the forces of the new cells. The process of mitosis/meiosis can be explained and shown as the formation and deformation of a magnetic field and considering a magnetic field as “a rule for operation”.

Cellular reproduction controls are the formation and breakdown of an internal magnetic field (bioelectromagnetic field) and can be represented by a poynting vector. Simply depicting a poynting vector the mutually orthogonal component is a poynting flux (magnetic component), intimately associated is an electrical charge. Electrical activity mediated thought the plasma membrane (Na/K) pump creating internal energy in the cystol Atp>Adp+Pi,. switched cdk/cyclin transcriptional DNA (GC AT) “nuclear polar territories”(55 Energy doc 7) stabilize the electrical energy

Within a cell the structural transition of DNA mediates functional changes in the permitivity of the cells' plasma membranes ionic channels resulting in cytsolic pH. change. DNA intracellularlly orchestrates.electrochemical gradients functioning to direct bioelectromagentic fields mechanistically controlling the cell. Viewing the cell as an electronic structure, simply as a battery, with controls being bioelectromagnetic. The capacitance of the cell (battery) electronic properties is based nuclear structural confirmations of the cellular DNA. (L. C. Confirmation). The highest ordered state of DNA is chromatin, the 3M model displays the mechanism.

DNA Liquid Crystals

The intrinsic structures of cellular DNA orchestrates biochemical synthesis using electrochemical,(electronic: ionic transduction across the plasma membrane and based in a tensgrity model(7), chemical: inducing cystolic pH. changes), the symmetry of the (electron) energy and current densities are controlled and displayed by the electrodynamics and conformational transitions of nuclear architecture DNA.(double helix, bead on string, lampbrush, chromatin). DNA intracellularly has four well know confirmations: double helix, bead on string, lampbrush, chromatin. These DNA confirmation have unique liquid crystal confirmations (l.c) to measure field strength of bioelectromagnetic field.

These confirmations change though out the cell cycle. The dynamics of the change can be associated and fixed with the cell cycle designation, G1, S, G2, M. Therefore G1>double helix, S>bead on string,, G2>lampbrush, M>, chromatin.

The “cyclic operating systems” of the cell cycle controls are a bioelectromagnetic field established by nuclear DNA transition in response to: Extra cellular ion concentration, plasma membrane,
cystoli????????????????????????????????????????????????????????????????????????????????????????????????????????????????????????????????hite blood cells (□HYPERLINK 1 “B9”□□9□). In contrast, here we employ capacitance measurements as a means of detecting and
qua????????????????????????????????????????????????????????????????????????????????????????????????????????????????????????????????e, in an applied low-frequency AC electric field, its polarization response, in combination with the motion of the surrounding c of nuclear architecture DNA.(double helix, bead on string, lampbrush, chromatin).

Ionic currents or “asymmetries of ionic flux”(4)though the plasma membrane H2O,(Na, K, Ca2+,Mg2+) act in response to ordered energy and upon intracelluar concentrations vary (flux) the nuclear (DNA) architecture during the cell cycle or “embryo patterning.”(4) Base pairing, AT, GC banding and functions of the electronic cell are sequence dependent.

Variable p.H. intracellular conditions (ATP/cyclins, ADP/cdk, AMP/cdk2) are controlled by the electrodynamics of the DNA structure with regards to the conductance and function of the cell.

The Bioelectromagnetic field is produced before replication intracellularly by the downward spiral of phospholations and the upward composition of DNA. The highest ordered state of DNA is chromatin at metaphase that displays the bioelectromagnetic mechanism.

Understanding DNA during replication/transcription by a balance of charge, DNA/RNAs the symmetry of energy is a magnetic force. In respects to physics “energy flux is intimately connected with linear momentum density and this is connected with angular momentum”(energy doc 7 theory page 4)

These DNA confirmations have unique liquid crystal confirmations (l.c) to measure field strength of bioelectromagnetic field. These can be applied to what I term as structural functional positional information(sfpi) that is displayed thought out the cell cycle and give rise to signaling pathways, environmental needs, within functional groups of cells (ex muscle, liver, nervous).

3)>the best direct evidence for it,

Could a magnetic field direct cell activity? Jim Valles has disrupted mitotic apparatus with emf (6)

The controls of the cell system are electronic. The magnetic component of the electrical system appears minor yet is the controlling dynamic. The intrinsic structure of DNA in function and structure directs electrochemical energies to electromagnetic field (Bioelectromagnetic). The spatial temporal characteristics of cellular DNA liquid crystal confirmation regulate cellular activity.

Electrogenetics describes the energy requirements, energy exchanges, and electronic circuits, which allow gene reactions to occur in the living state. Merrill Garnett which whom I have extensive correspondance with term Electrogenetics is the basis for designing medicines that can short circuit the electrical charges in cancer cells and produce their selective electrocution.

Experiment Creating and performing simple experiment one must see (charge mediation) macroscopic change in a cellular environment (microscopic). A single live cell mediating electrical charge. This was accomplished by growing yeast cells (eucararyotic) and staining them with pH. Indicators. By microscopic analysis pH. Indicators became incorporated into live cells. Visible structures included the extracellular matrix (halo), plasma membrane, cystol and nucleus.

Cells appear colored in respect to structures mentioned. This demonstrates pH. (H+, OH−) gradients or ionic current within one live cell. Cells were in all phase of the cell cycle via visual observation. The color changes ranged from pH 3.0-6.8 though the cells and are corresponded with their phase of cell cycle. The common change in pH were noted within the cell structure were large ionic flux noted in: extracellular matrix (halo), plasma membrane, cystol and nucleus, nuclear membrane. Average pH for all phases of cell cycle(G1,S, G2,M) were:.extracellular matrix (halo)=6.7 pH, plasma membrane=4.9 pH., cystol=pH3.4 nuclear membrane=pH4.7 and nucleus=5.0. I do not believe that ionic current has been shown though out a whole cells previous to this and noted structures functioning in such electrically dynamic way.

Further the cells from varying cell cycle stages showed change by the addition of 1 m. KCl. All cells appeared to be driven into G2 phase. The sodium/potassium pump was driven by the addition of K. the ionic flux induced by the addition of potassium shows change in intercellular ionic flux.

Levin

The experimental data being generated in Levins lab, takes major thoughts such as embryos (one cell) and is mechanistically showing the function of the flow of “asymmetries of ionic flux” the energy and control of right left asymmetry within genomic information. This work of bioelectrical controls in embryos displays the mechanism of control.

what experimental predictions it>makes (and how it is useful, and more useful than current theories),

Many studies indicate that cell function maybe electric with a few cite here. (4,5,6,7,8, 12,15)

The theory is novel. Current models and theory do not address many or any issues. The control of mitosis and the cell cycle are mostly explained by “mechanisms poorly understood.”.

Could a magnetic field direct cell activity? Jim Valles has disrupted mitotic apparatus with emf (6)

Nordenström presents an electrophysiological view of acupuncture. (15)Role of capacitative and closed circuit currents or “biologically closed circuits”;however, the circuits are not present as controlled by DNA function and are not viewed as open circuits cytosolic environment

Experiment predictions such as Levin Na/K asymmetries, were in fact predictable before the study had issued.

Bioelectromagnetic control Levin provides strong experimental evidence making the theory acceptable. A final draft of the theory was written last August/September and Levins work was issued sometime in October. Theoretically, proposing that during early in the cell cycle G1, a double helix confirmation would dominate nuclear structure, electronically function as a solenoid with response from AT banding to GC banding regions. The ionic flux via plasma memebrane into the cell would be sodium, potassium (ordered energy) and the driving energetic cytosolic environment ATP-cyclin.

Shows Goodman and Blank work regulations of genes response to emf.(12)

Genomic Function

It is obvious that genomes' function can be shown in what I term electrodynamic profiling

As the expirement shows electrical activity though the cell, It can be used as a method of evaluating cell systems and the genomic function. Provide vector analysis of cellular pathways. Create electronic signatures of biological systems. Development and improve drug interaction on a cellular level. Evaluate electromagnetic fields and there effects on cells creating new SAR standards. Translation and functionality of known and unknown genomic sequences for drug discovery. Allow computational analysis of electronic function of DNA. Help in neurological pathways and assessment.

Showing usage and commercial applicabilty. 9 12 03 Posting by Norvatis.

Epigenetics group within the Functional Genomics division. Projects will reflect our interest in identifying key regulators of chromatin structure involved in disease initiation and/or progression. The epigenetics group consists of four highly interactive labs housed at the new home of Novartis Institutes for BioMedical Research, Inc. in Cambridge, Mass. and is focussed on developing a therapeutic program based upon epigenetic mechanisms. how it could be falsified (what experimental observations would>definitively disprove your theory).

“DNA does not mediate a charge”(13)

If DNA can proven definitive not to mediate an electrical current or charge or magnetic component this would falsify the theory

Any studies that would should ac or dc field does not effect the cell or biological systems in regards to cell cycle control or cellular reproduction. The effects of a electrical, magnetic, or ionicly induced field do not change the field (bioelectromagnetic) strengths of the DNA nuclear architecture, liquid crystal properties. Which are predicated by the bioelectromagnetic theory.

Showing the mechanistic control of the biological cell as physical measurable entity of electric, magnetic, ionic currents can without difficulty be converted to harmonic (sound), photonic (light), vibrational, rotational measurables energies.

If ionic current did not exist though out cell as outlined in experiment.

A study that shows a cell does not have an electronic nature.

A magnetic field is not the controlling force of cellular division.

To prove rf do not interfere with biological systems.

Within a cell and groups of cells are in fact largely bioelectromagnetic and studies proving no such bioelectrical activity would disprove theory.

Studies that falsify would need to prove that there is spatial temporal relation between nuclear architecture and cellular controls. Realizing that electrodynamics of cell systems is largely understudied and the model that are present may not be one hundred percent accurate, yet they are as accurate and more expansive than studies or theory today.

The sheer enormity of information and scientific understanding required compiling and explaining such mechanisms has been solely complied

“Having reached the milestone of sequencing entire genomes, fundamental issues in understanding human biology are how genomes are organized in living cells and how gene expression programs are regulated. My laboratory seeks to uncover how nuclear architecture and genome topology affect genome function in living cells. The importance of nuclear architecture in controlled genome expression is evident from the critical role nuclear reorganization plays in stem cell differentiation, carcinogenesis and cloning by nuclear transfer. To gain insight into nuclear function in vivo, we are applying a multifaceted approach to study the biophysical properties of proteins in living cells, the spatial organization of genome within the cell nucleus and the application of imaging methods to study pre-mRNA processing events.

To understand the nuclear environment in which genomes are expressed, we are probing the biophysical properties of proteins and chromatin using in vivo imaging

Our cell biological studies of genomes and the cell nucleus are aimed at uncovering fundamental concepts of genome organization and nuclear function in vivo and they are providing opportunities for applying these principles to human disease diagnosis, therapeutics and bioengineering.”

Tom Misteli, Ph.D. Principal Investigator (14)

Fundamental concepts of genome organization and nuclear function in vivo are shown as bioelectromagnetic. That bioelectrical activity plays no role and bioelectromagnetic field does not exist. I believe that your studies of the functioning embyo and the patterning

Levines' Studies on embryos show the logical sequence of usage of genome information(4). Thus any studies disproving the Katp activity, right left patterning, electromagnetic control in morphogenisis, ionic conduction, etc. would disprove theory and understand presented herein.

Basic scientific understanding in a trinity of the hard sciences: biology, chemistry and physics leads to explanation. Any studies not solely based on thermodynamic which show nuclear function. We do not disregard thermodynamics yet the energy electronic is a lower requirement shown by Goodman with heat shock protein being downstream from ERME.

Theorizing on the appearance of a cell at metaphase the chromosomes appear held within a magnetic field, I propose that DNA creates a magnetic field controls cell division. Physics uses the term electrostatics to explain magnetic behavior. Electrostatics interactions serve in resolving the physical explanation of cell division.

A cell functions electronically. The pumps of the plasma membrane selectively uptake extra cellular elemental ions (Na/k. The balance of ion flux is regulated by DNA and Atp production or usage. The cell is an electronic structure. In a cell regulation of ionic current though the plasma membrane are determined by the structure of DNA within the cell.

The cell is in constant ionic flux except at meta phase and the mechanics of cell division are that of electromagnetic field A magnetic field controls the cell. The Magnetic field is produced by the intrinsic structure of the DNA molecule with in the cell. DNA structurally has four confirmed states and the range of capacitance is from non conducting to super conducting.

The cell during reproduction as birefringement of chromatin during prophase displays an electronic and mechanical relationship though out metaphase, teleophase, anaphase. The physics of this process can be explained by electrostatics(2), yet the process and understanding is static. The process of mitotic division is dynamic and an electrodynamic view (completes and explains the movement of a magnetic field.(3) The middle of cellular reproduction metaphase is a magnetic field.

?Ionic currents or “asymmetries of ionic flux”(4)though the plasma membrane H2O,(Na, K, Ca2+;Mg2+) act in response to ordered energy and upon intracelluar concentrations vary (flux) the nuclear (DNA) architecture during the cell cycle or “embryo patterning.”(4) Base pairing, AT, GC banding and functions of the electronic cell are sequence dependent.

Variable p.H. intracellular conditions (ATP/cyclins, ADP/cdk, AMP/cdk2) are controlled by the electrodynamics of the DNA structure with regards to the conductance and function of the cell.

The Bioelectromagnetic field is produced before replication intracellularly by the downward spiral of phospholations and the upward composition of DNA. The highest ordered state of DNA is chromatin at metaphase that displays the bioelectromagnetic mechanism.

The Bioelectromagnetic field is produced before replication intracellularly by the downward spiral of phospholations and the upward composition of DNA. The highest ordered state of DNA is chromatin at metaphase that displays the bioelectromagnetic mechanism.??

Understanding DNA during replication/transcription by a balance of charge, DNA/RNAs the symmetry of energy is a magnetic force??

Biology studies life and the simplest unit is a cell. The cell in structure and function is complex. The biochemical pathways of cell signaling change the structure of the cell. Structural transitions within the cell do change the functioning. The order of structural functioning transitions changes the way in which the cell directs energy. The currency of energy in biological system is adenosine triphosphate (ATP). The cell regulates the energy usage in a highly ordered behavior. ATP energy is coupled with four elemental ions which can make or use energy in a cell. The elemental ions react to ATP cleaving of a phosphate Transitions of ATP are ATP>ADP>AMP. The elemental ions react to compensate this energy transfer. These ions specifically react with DNA to elucidate the Order and complexity of cell function by looking toward the usage of the genomic information.

### REFERENCES

1)http://fig.cox.miami.edu/˜ddiresta/bi101/Cells.htm

2) www.crab.rutgers.edu/˜gagliard/webpre.pdf

3)http://micro.magnet.fsu.edu/electromag/java/mitosis/index.html

4)http://www.drmichaellevin.org

5) www.cancerci.com/content/2/1/3

6)http://www.physics.brown.edu/Users/Faculty/valles/VallesLabFiles/BFieldManipulation.htm

7) http://www.hms.harvard.edu/dms/bbs/fac/ingber.html

8) www.powerlinefacts.com/goodman-blank.pdf

9) Garnett proviate correspondance

http://www.electrogenetics.net/ http://www.electrogenetics.net/

http://www.electrogenetics.net/Biological_Liquid_Crystal_Theory.html

10) FF Beaker in private correspondence

11I)Hana Lin et al. Regulations of genes with electromagnetic response elements. Journal of Cellular Biochemistry 81:148 (2001)

13)Ben Farby, Harvard Magnetic Twisting Cytometry Lab (private discussion)

14)http://rex.nci.nih.gov/RESEARCH/basic/lrbge/cbge.html

http://rex.nci.nih.gov/RESEARCH/basic/lrbge/cbge.html□

15)Nordenström BEW: An electrophysiological view of acupuncture. Role of capacitative and closed circuit currents and their clinical effects in the treatment of cancer and chronic pain. Am J Acupuncture 17:105-117 (1989).

16) L. L. Sohn,*□HYPERLINK 1 “FN151”□□†□ O. A. Saleh,* G. R. Facer,* A. J. Beavis,‡ R. S. Allan,‡ and D. A. Notterman:‡□HYPERLINK 1 “FN151”□□†□Capacitance cytometry: Measuring biological cells one by one Departments of * Physics and ‡ Molecular Biology, Princeton University, Princeton, N.J. 08544 Proc. Natl. Acad. Sci. USA. 2000 September 26; 97 (20): 10687-10690

17)M. V. Sataric and J. A. Tuszynski: “The Impact of Regulatory Proteins on Nonlinear Dynamics of DNA,” submitted to Physical Review E.Web http://mitacs-

Copyright 2003 Anthony S Fuccione

The symmetry of genomic (DNA) responses in a cell though out the cell cycle The model of 3M shows

### BRIEF SUMMARY OF THE INVENTION

The invention uses the physics of an electromagnetic field which displays mechanistic controls a cell and reproduction biological cell. The processes of cell reproduction, called mitosis or meiosis, Object of invention

### Current Research

Electromagnetic (EM) fields have been used therapeutically for accelerated healing and pain control, but they have also been associated with adverse health effects. To understand these biological effects, we have been studying the interaction of low frequency EM fields with cells at both the cellular and molecular levels. Our studies with cells have shown that 60 Hz EM fields induce stress genes and stress response proteins in cells. The stress response is a protective mechanism induced by many potentially harmful environmental stimuli and characterized by the synthesis of specific proteins that assist the renaturation and transport of other proteins. Our studies suggest that EM fields initiate the stress response by interacting with electrons moving within DNA. We have identified a 900 base pair segment associated with the response to EM fields, that when removed eliminates the response, and when transfected into a reporter construct, causes the construct to become EM field responsive. We have also investigated the mechanism of EM field interactions at the molecular level through effects on three reactions, electron transfer in cytochrome oxidase, ATP hydrolysis by the Na,K-ATPase, and the Belousov-Zhabotinski (BZ) reaction (the catalyzed oxidation of malonic acid). The BZ reaction is studied with ordinary reagents, so there is no problem of impurities as with biological preparations. All three reactions show:

EM accelerates the reaction rate, i.e., electron transfer rate

EM competes with the chemical force, so its effect varies inversely with the reaction rate thresholds for interaction are low, comparable to levels found by epidemiology effects vary with frequency, and there are different optima for the reactions studied: ATPase (60 Hz), cytochrome oxidase (800 Hz), BZ (250 Hz)

These properties are consistent with the idea that EM fields affect many biological systems by interacting with electrons moving during redox reactions and also within DNA.

### DETAILED DESCRIPTION OF THE INVENTION

Best mode contemplated by me for carrying out the invention would be in application.

Academic

Certainly application by mean of academic acceptance would yield the greatest results as an academic teach tool. For biologist to explain cellular reproduction, for the physicist to have known “source charges” in measurement of electromagnetic field interactions, as the chemist to show the ionic, p.H. changes

Immediate clinical application can be imploded. Alteration of cell types and cell line in regards to biotechnological advancement are numerous deciphering the human genome appears to have the greatest benefit to man kind

Having strong belief in the system and methods of evaluation, a win win situation exists.

p.H changes thoughout (deferiation usage of chemical energy)

ION<DNA OF FREE P in system H2O ACID BASE RX redox

IONIC INTRODUCTION CURRENTS

Transitions of ATP are ATP>ADP>AMP. The elemental ions react to compensate this energy transfer.

DNA STRUCTURE (electrical and magnetic)

ELECTRONIC NATURE capacitance of the cell (battery) electronic properties is based nuclear structural confirmations of the cellular DNA

membrane capacitance and conductivity of mammalian cells, which reflect their surface morphological complexities and membrane barrier functions, respectively, have been shown to respond to cell physiologic and pathologic changes. Responce to physiologic change is regulated via nuclear architechure DNA

Understanding DNA during replication/transcription by a balance of charge, DNA/RNAs the symmetry of energy is a magnetic force. In respects to physics “energy flux is intimately connected with linear momentum density and this is connected with angular momentum”(energy doc 7 theory page 4)

symmetry of the (electron) energy and current densities are controlled and displayed by the electrodynamics and conformational transitions of nuclear architecture DNA.(double helix, bead on string, lampbrush, chromatin)

measures photonic emission. Biophotons are measuring photons herein we examine electronic usage. PLASMA MEMBRANE (include proteins G proteins)

membrane capacitance and conductivity of mammalian cells, which reflect their surface morphological complexities and membrane barrier functions, respectively, have been shown to respond to cell physiologic and pathologic changes. Responce to physiologic change is regulated via nuclear architechureDNA

Transitions of ATP are ATP>ADP>AMP. The elemental ions react to compensate this energy transfer.

DNA structure

electronic

Electronic nature

Physcial

Electrodynamics show higher symmetry in DNA structure

Nuclear region

Envelope mechanical function

plasma membrane,

physcial

polarization depolarization

Electronic

Ionic currents . . . NA/k

Cytosolic environment

Physical

pH.

Conductive fluid medium

Acid/ base

Electronic

Phosphate cdk,

ATP free pii in system

Acyletation, protonation

Cytoskeleton

Physical

Electronic signal though physical means

Mechanical intergins

celluar environment e

EMC

I claim:

1. A method of evaluation and profiling of electrodynamic interaction based on genomic response in cells creating devices and studying of energy and usage within the cell.
a. evaluation of any parts or functioning together as the genomic (DNA) function within the context of the cell and the known pathways of chemical and physical changes
b. conducting, using and controlling electronic, magnetic and photonic energy providing vectorial analysis of structures of DNA regulating cellular cycling pathways.
c. functioning of cells on the understanding of a bioelectromagnetic nature.

2. A method for evaluation and profiling of electrodynamic interaction based on genomic response in cells examining electronic nature of cells consists of describing, studying, detecting and quantifying electrochemical interactions of the cells' electronic energy from DNA developing a new physics clarifications examining chemical reactions utilizing cellular DNAs' higher symmetry electromagnetic as displayed by genomic function directing cells' electronics in a higher symmetry mechanism though physical explanation of chemical reactions directing technology based in biology, chemistry and physics, systems.

3. A method for evaluation and profiling of electrodynamic interaction based on genomic response in cells examining electromagnetic nature of cells and recording and charting active.
a. Creating electromagnetic signature of biological systems

4. A method for evaluation and profiling of electrodynamic interaction based on genomic response in cells examining electronic nature of biomolecules and recording and charting active.
a. Creating electronic signature of biological systems

5. A method for evaluation and profiling of electrodynamic interaction based on genomic response in cells examining electromagnetic nature of biomolecules and recording and charting active.

6. A method for evaluation and profiling of electrodynamic interaction based on genomic response in cells to establish a system based on the design principles of DNA genomic function within a cell recording and charting active.
a. for engineering applications which include systems which are: physical, mechanical, chemical, biological, mathematical, electronic, magnetic, energy, computing, software, data storage or any combination of above.
b. expressing system as: 1-dimensional, 2-dimensional, 3-dimensional, 4-dimensional, quantum, vector or as Hamiltonian or any combination of above system.

7. The method of claims 2, 3, 4, 5, and 6 for any electronic or magnetic fields or potentials in mapping, evaluating and using electric and magnetic field recording and charting active of cells.
a. imagining of cells or molecules interaction though chemical, electronic, physical means.

8. A method of evaluation and profiling of electrodynamic interaction based on genomic response in cells whereas the physiochemical properties of spatiotemporal organization of biomolecules regulating and functioning by nuclear DNA electronic structure.

9. A method of evaluation and profiling of electrodynamic interaction based on genomic response in cells designing DNA as harmonizing electromagnetic symmetry mechanism.
a. functioning of DNA 5′ terminus to 3′ or 3′ terminus to 5′ system of DNA
b. bi-directional electromagnetic flow of energy-(DNA bidirectionality or symmetry of energy flow) interpreting the input energy(chemical) to the charge coming time-domain cell cycle, and the output energy from the charge being emitted into 3-space, comprises a scalar potential as DNA scalar potential is measured by protein, amino acid, RNA(s) or DNA interaction, polyamines ex. Putricine, histone interaction
c. heterodimer transcriptional proteins which symmetrically bind due to electrostatic free energy palindromes of DNA sequences: those proteins in the class of fos-jun displaying the directionality mechanism

10. A method of evaluation and profiling of electrodynamic interaction based on genomic response in cells creating devices and studying displaying the enthalpy (Chemical) control of energy within a cell converting to entropy (free energy) storage or mechanical usage in biological molecules in spatiotemporal (cell cycle) organization.

11. A method of evaluation and profiling of electrodynamic interaction based on genomic response in cells consisting of: a biological cell or cells, chemical explanations of electron(ic) movement revealing the physics (al) and mechanical relationship though the cell cycle and reproduction and any portion thereof.

12. A method of evaluation and profiling of electrodynamic interaction based on genomic response in cells consisting of: a biological cell or cells, chemical explanations acid base reactions revealing the physics (al) and mechanical relationship though the cell cycle and reproduction and any portion thereof.

13. A method of evaluation and profiling of electrodynamic interaction based on genomic response in cells the chemical processes as electron transfers and the abilities of known and unknown molecules carrying, transferring, storing electrons at fixed points in time and in real time.

14. In regards to claim 11: Considering the biological structure of a cell as an electronic structure. consisting of a plasma membrane, a cytosolic environment, DNA magnetic force mechanical due to chemical gradients in a cell: nuclear envelope, DNA, plasma membrane, cytosol, extracellular matrix.

15. In regards to claim 11: A system based on the design principles of DNA genomic function within a cell defining ultimate design of DNA electromagnetic mechanical mechanism.

16. A method for evaluation and profiling of electrodynamic interaction based on genomic response cell function (in vivo, in vitro) applying a multi faceted approach to study the biophysical properties of biochemical interactions relative to the cells genome as the sequencing of the genome accomplishing function.

17. A method for evaluation and profiling of electrodynamic interaction based on genomic response cell using the electrostatic interactions of a magnetic field as the static interactions of a cell during metaphase of mitosis.
a. (FIG. 1.electromagnetic field interactions of DNA of a cell during reproduction during metaphase mitosis termed three m)
b. explaining an equilibrium of (electron) energy symmetry in a physical system of a cell.
c. Defining chromatin as the highest ordered state of cellular DNA, as an ideal crystal directing magnetic component of an electric field.
d. analyzing, quantifying and explaining symmetrical systems of electromagnetic interaction based on a cells using electronic and magnetic energy of genomic response.
e. defining of biological cell(s) relative to DNA function as bioelectromagnetic, electromagnetic, electronic, magnetic,
f. defining electromagnetic field interactions to biological systems
g. eliciting electronic or magnetic responses from a cell(s)
h. defining known or unknown energy usages of cellular composition of DNA.
i. Showing balance of shape size and position of cellular DNA in relation to cytosolic and extracellular matrix
j. Using a natural physical system of a cell during metaphase (FIG. 3) of cellular reproduction in showing, explaining quantifying equilibrium of DNA (Chromatin) symmetry in physical system such as magnetic and electrical fields.
k. using and explaining electrostatic interactions of cells during reproduction: mitosis/meiosis at metaphase being a magnetic field, termed three M, being a purely natural symmetrical system.
l. using the three M model to describe electrodynamics of electromagnetic field interactions within the biological system of a cell during anaphase, prophase, metaphase, in a cell and the deformation during telophase and cytokinesis as magnetic or electromagnetic force.
m. using the three M model to describe electrodynamics of electromagnetic field interactions within the biological system of a cell during reproduction and the life (cell cycle) of a cell
n. using and explanting of mechanisms and operation of magnetic or electrical interactions in the natural system of a cell.
o. Using classical and quantum physics to evaluate and quantify potentials, fields and waves in a biological cell system.
p. Using in explanation of mechanisms chemical, mechanical and physical and operation of cellular division in terms of magnetic or electrical interactions.

18. A method for evaluation and profiling of electrodynamic interaction based on genomic response in cells controls of cells regulated by nuclear electronic structure transitions characterization of activity of responses in a single cell, group of cells, organ, tissue or organism.
a. electron transfer mechanisms within DNA
b. changes in intracellular p.H. due to ionic flux though the plasma membrane
c. change in voltage due to dynamics of cytosol, extracellular, nuclear envelope, DNA structure.
d. fluid mechanics of cytosolic environment.

19. A method for evaluation and profiling of electrodynamic interaction based on genomic response in cells examining functioning as an electromagnetic exploring and defming electromagnetic properties, electronic, computing, capabilities of DNA confirmations within cellular environment.

20. A method for evaluation and profiling of electrodynamic interaction based on genomic response determining of gene and protein function expressing symmetry of the (electron) energy and current densities are controlled and displayed by the electrodynamics and conformational transitions of nuclear architecture DNA.(double helix, bead on string, lampbrush, chromatin)

21. A method for evaluation and profiling of electrodynamic interaction based on genomic response in cells which consists of describing, studying, evaluating and quantifying
a. usage of energy within the cell.
b. application of Physics of energy systems relative biological systems.
c. modeling and applying of biological cell(s) as a magnetic and electronic structures.
d. controlling cell cycle
e. interacting Cell-cell
f. communications of tissue and organ systems
g. patterning of embryo
h. effecting electromagnetic fields on organism

22. A method for evaluation and profiling of electrodynamic interaction based on genomic response in cells which consists of describing, studying, evaluating and quantifying usage of bioelectromagnetic control of cell(s) and mapping whole genomes according to electronic and magnetic values.

23. A method for evaluation and profiling of electrodynamic interaction based on genomic response in cells which consists of moving elements (known as pumps or channels) though the plasma membrane specifically ions of sodium, potassium, calcium, magnesium, chlorine and water (H+, OH−).
a. explaining and using ionic currents across plasma membrane
b. explaining and using ionic transduction across plasma membrane
c. explaining and using ionic changing of conduction of plasma membrane
d. explaining and using ionic currents changing ionic currents
e. explaining and using ionic currents in the gap junctions
f. explaining and using the electrogenic nature of plasma membrane

24. A method for evaluation and profiling of electrodynamic interaction based on genomic response in cells which consists
a. Changing and varying of pH. intercellular environment and conductivity of plasma membrane
1) changing Plasma membranes ionic channels the physical and chemical nature of the fluid within the cell
2) changing Plasma membranes ionic channels the physical and chemical nature of the plasma membrane.
3) changing Plasma membranes ionic channels the physical and chemical nature of the cytoskeleton.
4) changing Plasma membranes ionic channels the physical and chemical nature G-protein-coupled
5) changing Plasma membranes ionic channels the physical and chemical nature of nuclear region of cell
6) changing Plasma membranes ionic channels the physical and chemical nature of nuclear envelope
7) changing Plasma membranes ionic channels the physical and chemical nature of cellular DNA
8) changing Plasma membranes ionic channels the physical and chemical nature of cellular DNA shape or size
9) Receptors changing plasma membranes ionic channels the physical and chemical nature of the interior of cell

25. A method for evaluation and profiling of electrodynamic interaction based on genomic response in cells which consists
a. Changing permeability of plasma membrane
b. Polarization of plasma membrane
c. Depolarization of plasma membrane.
d. explaining and using ionic currents as mechanically changing structural integrity of architecture structure

26. A method for evaluation and profiling of electrodynamic interaction based on genomic response in cells which consists
a. transitioning the conformations of DNA within the cell
b. transitioning the conformations of nuclear architecture
c. measure field strength and functionality to create cellular change due to electromagnetic properties of DNA.
d. conducting, non conducting, relays, capacitors, currents, magnetic flux densities
e. assembling and self assembling of nano-scale DNA circuit

27. A method for evaluation and profiling of electrodynamic interaction based on genomic response in cells which consists
a. Defining, using and associating changes with a cells' cycle designated as GO, G1, S, G2, M relative to the geometry and or architecture of DNA
b. Defining, using and associating the cell's cycle as a cyclic operating system relative to the geometry and or architecture of DNA
c. Defining, using and associating the cell's cycle as a cyclic operating system and any and all parts thereof relative to the geometry and or architecture of DNA
d. Defining, using and associating changes with a cells' cycle, embryo patterning, cellular responses within an organism relative to the geometry and or architecture of DNA
e. Defining, using and associating the cell's cycle as a cyclic operating system and any and all parts thereof relative to the geometrical complexes of DNA
f. Defining, using and associating the cell's cycle as a cyclic operating system and any and all parts thereof relative to the architecture of DNA

28. A method for evaluation and profiling of electrodynamic interaction based on genomic response in cells which consists cytoskeleton, nuclear pore, nuclear complex, geometry, architecture or structural integrity.

29. A method for evaluation and profiling of electrodynamic interaction based on genomic response in cells in tracking, using, explaining, outlining microtubules and microtubule associate protein (MAP) reacting and sensing the bioelectromagnetic field.
a. measurement of magnetic force via electrical activity of microtubules, named dynein and kinesin.

30. A method for evaluation and profiling of electrodynamic interaction based on genomic response in cells which consists cell content of deoxyribonucleosides triphosphates:diphosphates: or monophospates relative to cyclin, cyclin dependent kinases or cyclin dependent.kinases 2 concentration. a-s.s listed and using (ase) ex ATPase(s)
a. ATP to cyclin concentration
b. ADP to cyclin dependent kinases (cdk) concentration
c. AMP or cyclic AMP to cyclin dependent kinase 2(cdk2) concentration
d. ADP to cyclin concenttation
e. AMP or cyclic AMP to cyclin concentration
f. ATP to cyclin dependent kinases (cdk) concentration
g. AMP or cyclic AMP to cyclin dependent kinases (cdk) concentration
h. ATP to cyclin dependent kinase 2(cdk2) concentration i. AMP or cyclic AMP to cyclin dependent kinase 2(cdk2) concentration
j. GTP to cyclin concentration
k. GDP to cyclin dependent kinases (cdk) concentration
l. GMP or cyclic AMP to cyclin dependent kinase 2(cdk2) concentration
m. GDP to cyclin concentration
n. GMP or cyclic AMP to cyclin concentration
o. GTP to cyclin dependent kinases (cdk) concentration
p. GMP or cyclic AMP to cyclin dependent kinases (cdk) concentration
q. GTP to cyclin dependent kinase 2(cdk2) concentration
r. GMP or cyclic GMP to cyclin dependent kinase 2(cdk2) concentration
s. TTP to cyclin concentration
t. TDP to cyclin dependent kinases (cdk) concentration
u. TMP or cyclic AMP to cyclin dependent kinase 2(cdk2) concentration
v. TDP to cyclin concentration
w. TMP or cyclic AMP to cyclin concentration
x. TTP to cyclin dependent kinases (cdk) concentration
y. TMP or cyclic AMP to cyclin dependent kinases (cdk) concentration
z. TTP to cyclin dependent kinase 2(cdk2) concentration
aa. TMP or cyclic TMP to cyclin dependent kinase 2(cdk2) concentration
bb. CTP to cyclin concentration
cc. CDP to cyclin dependent kinases (cdk) concentration
dd. CMP or cyclic AMP to cyclin dependent kinase 2(cdk2) concentration
ee. CDP to cyclin concentration
ff. CMP or cyclic AMP to cyclin concentration
gg. CTP to cyclin dependent kinases (cdk) concentration
hh. CMP or cyclic AMP to cyclin dependent kinases (cdk) concentration
ii. CTP to cyclin dependent kinase 2(cdk2) concentration
jj. CMP or cyclic CMP to cyclin dependent kinase 2(cdk2) concentration
kk. UTP to cyclin concentration
ll. UDP to cyclin dependent kinases (cdk) concentration
mm. UMP or cyclic AMP to cyclin dependent kinase 2(cdk2) concentration
nn. UDP to cyclin concentration
oo. UMP or cyclic AMP to cyclin concentration
pp. UTP to cyclin dependent kinases (cdk) concentration
qq. UMP or cyclic AMP to cyclin dependent kinases (cdk) concentration
rr. UTP to cyclin dependent kinase 2(cdk2) concentration
ss. UMP or cyclic UMP to cyclin dependent kinase 2(cdk2) concentration
1) alternately phospholating of protein in the class of p53,p21, rb proteins.

31. A method for evaluation and profiling of electrodynamic interaction based on genomic response in cells responding to anion and cation-pi interactions with responding to intercellular ions or chemicals specific ions of sodium, potassium, calcium, magnesium, chlorine and water (H+, OH−) to interacting molecules.

32. The method according to claim3O, using energetic base nucleosides which carry or store phosphate combining 31, using element and their ion(s) which are transported though plasma membrane, for evaluation and profiling of electrodynamic interaction based on genomic response in cells example (A)TP hydrolysis by the Na,K-ATPase

33. A method for evaluation and profiling of electrodynamic interaction based on genomic response in cells controlling of cells regulating nuclear electronic structure transitions
a. controlling of electron transfers due to hydrogen (electron) bonding of purines and pyrimidines
b. controlling of electron transfers of nucleic acid bases
c. controlling of electron transfers base pairs
d. controlling of electron transfers in base pairing
e. controlling of electron transfers in AT or GC banding
f. controlling of electron transfers in sequences of DNA
g. controlling of electron transfers in a sequence dependent manner using “double helix”structure
h. controlling of electron transfers in using “bead on string” structure
i. controlling of electron transfers in using “lampbrush” structure
j. controlling of electron transfers in using “chromatin” structure

34. A method for evaluation and profiling of electrodynamic interaction based on genomic response in cells which consists defining and using conformational states intercellular DNA
a. using “double helix” structure
b. using “bead on string” structure
c. using “lampbrush” structure
d. using “chromatin” structure

35. The method of claim 34 physical structure of cellular DNA describing, evaluating and using of these transitions as circuit element both partial and full functioning circuits and electromagnetic function.

36. The method of claim 34 and 35 physical electronic structure combining claim 32
a. using and creating virtual and actual structures of DNA and those observable structures to use exact correlation between them.
b. directing engineering application of this electromagnetic mechanism
c. making integrated circuits using DNA molecules as a support structure with methods also for making DNA based transistors, capacitors, inductors, conductors, relays diodes and battery design.

37. The method of claim 34 in defining and using the structures which are mixed as part and factors of the genomic structure exists as: solely: a: b: c. or d. or factors of: a and b: a and b: a and c: a and d: a,b and c: a,b and d: a,b,c and d: a,c and db,c: b,c and d: b,d: c,d

38. The method of claim 34, 37 in defming and using the structures interactions as liquid crystal.
a. defining the liquid crystal structure of DNA confirmation
b. evaluating electronic, magnetic and physical confirmations
c. showing controls regulated by DNA and nuclear structure transitions as electrical and or magnetic activity
d. using palindromes of DNA sequences genomic function explain bidirectionality of current or magnetic flux or non functionality of charge to mass ratio forces.
e. measuring and using asymmetries of ionic flux to explain symmetry of DNAIRNA replication or transcription as a response to physiologic change is regulated via nuclear architectures DNA
f. explaining understanding DNA during replication/transcription by a balance of charge, DNA/RNAs the symmetry of energy is a magnetic force

39. The method of claim 33 measuring, predicting, quantifying, and defining and evaluating electromagnetic properties of histone incorporating DNA sequences in the functionality of genomic response.
a. measuring Mediation of charge within the DNA molecule as histone pack DNA
b. measuring of charge mediation to the functional packing of DNA allowing access to information within the genome.
c. measurement and usage of new packing properties of DNA
d. using specific binding of CENP NH2 terminal domains sequence dependent
e. interactions of histone tail mostly NH2 and COOH switching

40. The method of claim 36 to predicting, evaluating electrical activity chemical intracellular cytosolic induction of ionic flux, and change in conduction of plasma membrane using chemical dyes or imagining devices showing electronic activity or electron or photon transfer.
a. the method of claim of 34 staining and sequential fluorescence analysis of the dyes bound to specific base regions and intercalating sites on DNA fluorescence intensity of each dye is proportional to the relative number of specific base regions or intercalating sites
a. active genomic regions
b. groups of genes
c. single gene
d. DNA/DNA replication (active or inactive)
e. DNA/RNA—snRNa, m-RNA, t-RNA
f. DNA/Protein
g. DNA/Amino acid
h. DNA/DNA polymerase
i. DNA/RNA polymerase
j. DNA/ion
k. Ion/protein
l. Protein/protein
m. Gene/protein
n. Protein/RNA
o. RNA/protein
p. RNA/ion
q. RNA/Amino acid
r. RNA/DNA
s. RNA/RNA
t. Amino acids
b. claim of 40 a using imagining devices detecting transfer or change of electronic or magnetic components
a. active genomic regions
b. groups of genes
c. single gene
d. DNA/DNA replication (active or inactive)
e. DNA/RNA—snRNa, m-RNA, t-RNA
f. DNA/Protein
g. DNA/Amino acid
h. DNA/DNA polymerase
i. DNA/RNA polymerase
j. DNA/ion
k. Ion/protein
l. Protein/protein
m. Gene/protein
n. Protein/RNA
o. RNA/protein
p. RNA/ion
q. RNA/Amino acid
r. RNA/DNA
s. RNA/RNA
t. Amino acids

41. A method for evaluation and profiling of electrodynamic interaction based on genomic response in cells regards static to dynamic transition hydrations of biomolecules electron transport using and examining single electron transfer of terminus:
a. denoted by positive (+) Amino (N) chemical depicted as NH3 or as a functional Amino group NH2
1) modifying by oxidations or reductions called a base chemically existing in forms of NH3, NH2, NH, N(−)
b. denoted by a negative (−)Caryboxyl (C) chemical depicted as COOH or as functional hydroxyl group —OH
1) modifying by oxidations or reductions called an acid chemically existing in forms of COOH, COO, CO
c. Combining a and b p.H. dependent structures
d. defining a and b as p.H. dependent structures reactive as claims 2, 3, 4, 5, 6, 24, 26,33,40
e. associating change with temperature

42. The method of claim 41 resulting upon and single electron transfers in DNA
a. Intercellular Terminus hydration and conduction electron transport.
1) 5′ and 3′ terminus of DNA molecule conduct current in opposing direction with a symmetry of magnetic force.
2) 5′ terminus to 3′ or 3′terminus to 5′ mimic the system of DNA electromagnetic and or any part thereof described within this application for use to use, build, design, regulation of know mechanical, electronic, computer programs, make known and identify biological pathways with a cell, groups of cells, organs, tissue, or organism for desire applicable results
b. A method of cellular evaluation based on the internal magnetic force exerted and magnetic energy directed within a cell via DNA chromatin leading to it most compact form, during cellular reproduction (mitosis or meiosis) flowing energy responsible for cellular division and DNA relative to step (e) in method 41 to express super conduction properties of DNA as an ideal crystal.

43. The method of claim 30, are specific to phospholation or de phospholation any cellular molecules ability including acetylations, methylations, deacetylations, protonation, deprotonation. a. redefining claims 24 (a) parts (3),(4),(6),(9).

44. The method of claim 40 in using a cell as a model or in actuality of conductance, fluxing or storing charge as a capacitor of cellular DNA and interaction in design or usage in magnetoelastic and or magnetostrictive device.

44. The method of claim 8 application of cellular mircoarray, disease diagnosis, drug discovery, pharmacogenomics and therapeutic responses to drugs, chemical elements, vibrations, light, electromagnetic fields, electric field, thermodynamics, or force.

45. A method for evaluation and profiling of electrodynamic interaction based on genomic response in cells which consists of describing, studying, evaluating and quantifying molecular protein motors
a. conversion of chemical energy into mechanical forces due to electrodynamics of DNA of actin, microtubules, dynein and kinesin motors.
b. Cytoskeleton

46. A method claims for evaluation and profiling of electrodynamic interaction based on genomic response in cells which consists of describing, studying, evaluating and quantifying microtubules and actin filament polarization as functional dependent.

47. A method for evaluation and profiling of electrodynamic interaction based on genomic response in cells which consists of describing, studying, evaluating and quantifying (chemical energy into mechanical force) electromechanical interactions
a. cytoskeleton
b. actin, myosin, intergins, Cytohesins, mircotubules, dynein and kinesin motors.
c. Measurement of twisting forces
d. Measurement packing forces
e. Measurement of rolling forces
f. Measurement of stress and or strain forces

48. A method for evaluation and profiling of electrodynamic interaction based on genomic response in cells which consists of describing, studying, evaluating and quantifying bioelectromagnetic field interactions of intercellular voltage changes, conduction of ions, p.H, gradients based on mechanical structures to modify cells.

49. A method for evaluation and profiling of electrodynamic interaction based on genomic response in cells establishing the biology of a cells' physical and fluid components and structures as measurable in physics.
a) equating electrochemical gradients potential to electromotive potentials
b) measuring electrochemical gradients to electromotive work

50. A method for evaluation and profiling of electrodynamic interaction based on genomic response in a cell establishing a bioelectromagnetic mechanism through systems of: a biological cells' DNA transitions, chemical reactions of biomolecules electron transfers, and the physics of electrodynamics defining magnetic field interaction.
a) using a bioelectromagnetic interaction
b) using a bioelectromechanical interaction

51. The method of claim 50 giving diagnostic analysis will give a measurable value electrodynamics and bioelectromagnetic resolution of normal functioning cells relative to desired functioning cells valves determining against know and unknown genes and sequences, including DNA base pair interaction fit the electrodynamical profile for those cells.

52. The method of claim 50, determining nature of a cell electromagnetic(s), electrodynamics, DNA electronics.

52. The method of claim 50 showing known and unknown regions within the chromatin, nuclear DNA(genes, sequences, bases pairs) will be analyzed for structural function on a singular cellular and a multicellular level with their context (SPFI) organ/tissue position which determining rates of cell reproduction are indicative of veracity of disease/DNA damage.

53. The method of claim 50, evaluating by electrodynamic profiles cells reproductive rates will be shown as high or low electrodynamical cell or cell types activity.

54. The method of claim 50. creating and establishing new standards of specific absorption rate (SAR) measured on the rate of energy absorption in a tissue by cell evaluation.
a. explore the effects of electrical and magnetic field producing on a cellular and genomic level for “safety” of these devices testing effects of RF energy on the body via cellular level of known and unknown hazards associated with RF energy exposure.
b. measuring tissue and organs on a cellular response both inter cellular and extra cellular relative to gene interaction and regulation
c. assessing quantity of energy and quality(kind) of energies which interfere with normal operation of cell and the control of cycle and reproduction.
d. effects of cells differs and would need to be examined for each tissue region relative to its location for accurate “scientific acceptable benefit”.

55. The method of claim 50 modifying of cells are to be used for therapeutic effects for all diseases introducing new cells within biological systems for desired proper function from stem cell as stem cell is a generic term for cells, electronic structures, which have not yet received information of their use and or function.

56. The method of claim 51, designing and use cell based biosensor.

57. The method of claim 51, mechanism of drug interaction with biological systems.
a. increasing efficient delivery of drugs to biological systems
b. solely increasing uptake of drugs
c. evaluating drug, anti-motoitics, inteterferons, oncogene-based cancer therapy, cytokines, platinum and other elements (ionic or elemental), antisense drugs, tumor suppressor enyzymes-p53,p-21 etc, antiangiogensis factors, DNA and RNA cleavage compounds on whole genomic response, cellular response, tissue and organ response relative to electrodynamic nuclear activity.

58. The method of claim 51 using radio frequency, electroporation and proton therapy or other electric or magnetic stimulation which may be solely. or in combination to enhance drugs or ions delivery and may encompass gene therapy.

59. The method of claim 51 assessing therapeutic responses to drugs, elements, vibrations, light, electromagnetic fields, electrical field, thermodynamics, force, with genomic information intact within a cell, as to specific DNA (in every biological systems differs if it be a single base difference or millions of bases in turn structural function positional information) displaying critical functioning.

60. Method of explaining effects of electromagnetic spectrum interactions with biological system using electronic/magnetic regulation of the cell cycle control as cells respond to limited controls of bioelectromagnetic fields they create and the electrodynamics of bioelectromagnetic control.

61. A method for creating a device to specification of a single biological cell or any portion of the pathway of the electronic cell function and may multi function as a battery to dynamically or statically store charge and responds to energy needs and conditions fueling by simple elemental ions as hydrogen fuel cells driven by complex biomolecules.
a)using the biological cell holding DNAs properties of conductance, fluxing the capacitance, storing charge to illicit response or shear usage of energy as magnetoelastic and magnetostrictive forces produced via these properties.

62. A method using the known and unknown liquid crystals of DNA for industrial application from nano-scale micro computing, energy usage, and energy production and encompassing simple circuit to magnetoelastic devices.
a) enhancing known circuits, computer processors, amplification of electrical, magnetic current, and sound, radio, microwave and light(uv, visible, IR, FTIR) frequencies thought the use or incorporation of liquid crystal from structure to function.

63. A method of using bioelectrodynamics to evaluate eastern and western medicine together.
a. Acupuncture and meridian system,chaka,aura etc. are known and based in electrical activities of cell with the philosophy is to disrupt the electrical energy of the system to return the cells to a “normal” state of function.
b. provide a base of integration of practical usage of eastern and western medical systems

64. The method of claim 52 stimulating of the brain or nervous system to elevate physical, emotional and psychological abnormalities
a)Using a machine that produces sound and electromagnetic frequencies can shown to direct cellular response
b)delivering precise frequency the machine to the brain or neutral network directly or indirectly to correct mis frequencies within the tissue or at points applied (chakas, merdian point) to particular regions of the cells of brain or nervous system responses to the energy should function to correct physiological, psychological abnormalities.

65. The method of claim 64 PRACTIONERS DIAGNOSITIC RESEARCH AND DELIVERY DEVICE for medical/biological application to evaluate and analysis of genomic information of desire cells line, type, tissue and organ system.
The device consist of four major components:
a) computer interface,
b) drug/proton delivery system,
c) a wave/proton function analyzer/harmonic translator
d) application by a applicator,a device or a hand tool, which direct energy and or drugs to subject or patient
1) Computer: the computer will be the interfacing device for the other elements of the machine.
2) The computer will have the ability to create records for patients which give and electrodynamic profile of effected/probed area.
3) Storing and create a profile of patient.
4) The computer will use a software package to determine structural functional positional information of cell(s) based on the electrodynamics of genomic information.
5) evaluating the information and determine valves of normal functions cells to abnormal functions and posses the ability to use a real time analysis of DNA usage.
6) determining and use valves(numerical) deliver precise aliquots of desired physical energy(emf, sound, light, chemical, or biological, drug, dyes to determine, correct, eradicate electrodynamics of cellular response.
a)These are to included signaling of cells both singularly and multi cell signals though a resonate analysis. Cell(s) resonating at frequencies the programs (software) can amplify signals received and signals can be desired physical energy (heat, emf, sound, light), chemical, or biological. The signals can be in response to induced energy as aforementioned and to drugs, dyes.
7) stimulating and evaluating responses of Drug/proton delivery system consist of pumps.to deliver desire ions, chemicals, drugs, dyes in liquid or dried from into the probed area.
8)providing information to the computer of p.H. valves, heat, and conductive valves of probed area as the drug/proton delivery system connected to the computer and the hand tool. The pumps within the system are driven by air and/ or liquid. This will also function and be made on the principles of a p.H. meter.
9) delivering wave/proton function analyzer/harmonic translator. To frequencies of light, sound, emf, electron, proton or magnetic to stimulate and evaluate response of probed area the translator connecting to the computer and applicator/ hand tool.
a translator may consist of laser(light), electrical source (able to deliver both electrical and magnetic sources of energy, frequency of sound source and/or radio waves.
10) Delivering system of Applicator/Hand tool for wave/proton function harmonic translator and drug deliver interfacing with computer system
a)Applicator/a hand tool may consists of needles three as three systems incorporated 1) wave/proton function analyzer/harmonic translator 2) drug/proton delivery 3) computer using needles as wires to determine electronic valves of the cells within the probed region.
b)The needles may triangulated or linear depend upon application. The needles are hollow and can be used to delivery of cells being examined. Inserting the needles are to desired area, tissue layer using as a probe and computer determines the tissue and the cell electrical activities as physical determining the reference of cell position and activity.
11). The bioelectromagnetic signature of cell function is to be determined, evaluated and corrective stimulation can be administered.

66. The method of claim 64, evaluation and profiling of electrodynamic interaction based on genomic response in cells designing supercomputers, optical systems, imaging systems, hydrogen fuel cell.

67. The method of claim 64 staining cells to show electrical activity.
a. live cells
b. dead cells
c. addition of chemical agents
d. addition of biological agents
e. addition of physical constraint
f. combination of any of the above
